(19) United States
(12) Patent Application Publication
(10) Pub. No.: US 2026/0053722 A1
(43) Pub. Date: Feb. 26, 2026

(54) Solid Compositions Containing Amine, Protonated Amine or Quaternary Ammonium Compounds

(71) Applicant: Stepan Company, Northbrook, IL (US)

(72) Inventors: Dennis S. Murphy, Libertyville, IL (US); Kathryn E. Merwin, Palatine, IL (US); Teresa Germain, Orland Park, IL (US); Aaron Sanders, Houston, TX (US); Sangeeta Ganguly-Mink, Chicago, IL (US); Sebastian Flieger, Elk Grove Village, IL (US)

(21) Appl. No.: 19/374,786

(22) Filed: Oct. 30, 2025

Related U.S. Application Data
(63) Continuation of application No. 17/974,189, filed on Oct. 26, 2022.
Application No. 17/974,189 is a continuation of application No. PCT/US2021/029112, filed on Apr. 26, 2021.
(60) Provisional application No. 63/017,362, filed on Apr. 29, 2020.

(51) Int. Cl. A61K 8/41; A01N 33/12; A01N 37/12; A01N 37/20; A01P 1/00; A61K 8/02; A61K 8/73; A61K 8/92; A61Q 5/12; C09K 8/54; C11D 3/00; C11D 3/20; C11D 3/22; C11D 3/30

(52) U.S. Cl. CPC A61K 8/416; A01N 33/12; A01N 37/12; A01N 37/20; A01P 1/00; A61K 8/0216; A61K 8/73; A61K 8/922; A61Q 5/12; C09K 8/54; C11D 3/001; C11D 3/2086; C11D 3/222; C11D 3/30; C09K 2208/32; C11D 2111/12

(57) ABSTRACT

Water-dispersible solid compositions that comprise at least one water-soluble polysaccharide, and at least one quaternary ammonium compound, amine, or protonated amine compound as an active ingredient that is absorbed by the polysaccharide are disclosed. The compositions are useful for a wide variety of applications, such as fabric softener compositions, hair care compositions, sanitizing compositions, and well bore treatment compositions.

### CROSS-REFERENCE TO RELATED APPLICATIONS

This application is a continuation of U.S. application Ser. No. 17/974,189, filed Oct. 26, 2022, which is a continuation of and claims priority to PCT Application No. US2021/029112, filed Apr. 26, 2021, which claims priority to U.S. Provisional Application No. 63/017,362, filed Apr. 29, 2020. The entire specifications of the applications referred to above are hereby incorporated by reference.

### FIELD OF THE INVENTION

The present technology relates to water-dispersible solid compositions that comprise at least one water-soluble polysaccharide, and at least one amine, protonated amine, or quaternary ammonium compound as an active ingredient that is absorbed by the polysaccharide. The compositions are useful for a wide variety of applications, such as fabric softener compositions, hair care compositions, sanitizing compositions, and oilfield well bore treatment compositions.

### BACKGROUND OF THE INVENTION

Recently there has been an increased interest in more environmentally sustainable and eco-friendly products. Products with a higher concentration of active ingredients and less water are desirable since they typically require less packaging, and therefore have a smaller environmental impact due to, for example, reduced transportation costs and less waste production. There is also a trend to formulate products with ingredients that are based on renewable resources derived from plants or animals, rather than fossil fuels. Such ingredients are considered “green” or “natural”, since they are derived from renewable and/or sustainable sources. As a result, they are more environmentally friendly than ingredients derived from fossil fuels. An ingredient having a high Biorenewable Carbon Index (BCI), such as greater than 80, indicates that the ingredient contains carbons that are derived primarily from plant, animal or marine-based sources.

Amines, such as esteramines or amidoamines, and quaternary ammonium compounds are valuable components for a wide variety of end uses, including fabric treatment, hair conditioning, personal care (for example liquid cleansing products), antimicrobial compositions, agricultural uses, and oilfield applications. Such compounds can be at least partially derived from biorenewable sources, which are desirable from an environmental standpoint. However, formulating such compounds into concentrated liquid compositions can result in products that may not be stable upon storage, especially when stored at high temperatures or at freezing temperatures. Instability can manifest itself as thickening of the product upon storage, even to the point that the product is no longer pourable.

Another problem with concentrated liquid compositions is that they typically require a solvent in order to achieve acceptable concentrated aqueous dispersions. The addition of a solvent is also usually required in order to have a product that has a low enough viscosity in its molten state that it is able to be pumped with conventional equipment. The added solvent is usually a volatile organic compound (VOC), such as isopropanol or ethanol, which is undesirable from an environmental standpoint. Moreover, stricter regulations limiting VOCs have been proposed, making it important to limit or eliminate solvents that contribute VOCs.

Because many alkyl quaternary compound are hydrophobic, they are often poorly soluble in water by themselves. Previous attempts to make water dispersible concentrated solid quaternary or amine formulations have drawbacks. For example, EP 111074 uses a silica to carry the quat, which has the disadvantage of bulking up the product and potentially leaving a residue, since silica is water insoluble.

WO 92/18593 describes a granular fabric softening composition comprising a nonionic fabric softener and a single long alkyl chain cationic material. However, the specification teaches that effective cationic softening compositions when used in granular form exhibit poor dispersion properties.

Solid, puck-type softener compositions are known for use in industrial and institutional applications (e.g. US 2014/0115794). These compositions are dosed by flowing a relatively significant amount of water over the puck and a small amount of material is solubilized. The compositions tend to intentionally be quite water insoluble (so too much material does not dissolve in a single softening cycle). Such solid compositions would not allow enough softener to get into a domestic consumer washing machine for adequate softening when added to the automatic dispenser drawer of a high efficiency washing machine, or to the center post (even if water were added along with the solid) softening dispenser of a conventional, non-high efficiency machine. In analogous fashion, not enough amine, protonated amine or quat would solubilize to be effective from these puck-type compositions in other applications such as hair conditioning or oilfield. Grinding them to a powder would not increase solubility since the compositions inherently have low water solubility.

Solid biocidal quaternary compositions are known for use in industrial and institutional applications. Preparation of these compositions are typically energy intensive. Often urea is used as a carrier for the biocidal quaternary to generate the solid form, which can be flakes or prilled. These forms are further made into tablets, pastilles, pucks or sticks. When used in water treatment for germ control, these urea containing compositions can result in an unpleasant amine or ammonia odor.

There is a need in the art for solid, concentrated amine, protonated amine or quaternary ammonium-containing compositions that are easily dispersed in water, and which leave no residue after being used in the desired application. It would also be an advantage to have a solid composition that is flowable.

### SUMMARY OF THE INVENTION

One aspect of the present technology is directed to a water-dispersible solid composition that comprises (a) from about 30% to about 95% by weight of at least one polysaccharide, (b) from about 5% to about 70% by weight of at least one amine, protonated amine, or quaternary ammonium compound, and (c) optionally, 0 to about 30% by weight of a solubilizer, wherein the amine, protonated amine, or quaternary ammonium compound has at least one alkyl chain of 10 carbons or greater, and is absorbed by the polysaccharide; with the proviso that, when the water-soluble polysaccharide is not agglomerated, the amount of the quaternary ammonium compound, amine, or protonated amine in the solid composition is not greater than 20% by weight, and when the water-soluble polysaccharide is agglomerated and has an aerated bulk density in the range of 250 g/L to 600 g/L, the amount of the quaternary ammonium compound, amine, or protonated amine in the solid composition is not greater than 35% by weight.

In another aspect, the present technology is directed to end use products that can be formulated with the solid composition, including hair care repair and conditioning compositions, fabric care compositions, antimicrobial compositions, and oilfield compositions.

In a further aspect, the present technology is directed to a method of making the solid water-dispersible composition.

### BRIEF DESCRIPTION OF THE DRAWINGS

FIG. 1 is a graph showing the corrosion rate profile for a solid composition of the present technology.

### DETAILED DESCRIPTION OF THE PREFERRED EMBODIMENTS

While the present technology will be described in connection with one or more preferred embodiments, it will be understood by those skilled in the art that the technology is not limited to only those particular embodiments. To the contrary, the presently described technology includes all alternatives, modifications, and equivalents as may be included within the spirit and scope of the appended claims.

“Biorenewable Carbon Index” (BCI) refers to a calculation of the percent carbon derived from a biorenewable resource, and is calculated based on the number of biorenewable carbons divided by the total number of carbons in the entire molecule.

“Biorenewable” is defined herein as originating from animal, plant, or marine material.

“VOC” refers to volatile organic compounds. Such compounds have a vapor pressure of greater than 2 mm Hg at 25° C., less than 7 carbon atoms, and a boiling point at atmospheric pressure of less than 120° C.

The compositions of the present technology are solid compositions that comprise one or more polysaccharides, and at least one amine, protonated amine, or quaternary ammonium compound that is absorbed by the polysaccharide. In some embodiments, the compositions of the present technology further comprise one or more solubilizers. The compositions can be in the form of a powder, or pressed into a tablet or other solid forms. Surprisingly, the compositions are readily dispersible in water, even at concentrations of amine, protonated amine, or quaternary ammonium compounds as high as 30% by weight or more.

### Amines

Amines that can be used in the present technology include secondary and tertiary amines such as esteramines and amidoamines. Tertiary amines are especially preferred. When amines are used in the composition, it is desirable to have the pH of the (aqueous) system the composition is introduced to for a given application at a value wherein the amine will protonate. Typically, the pH should be below 9, preferably below 8 and even more preferably, below 7.

### Esteramines

The esteramines of the present technology can be prepared by combining a natural oil or other fatty acid source and an alkanolamine, typically at a starting temperature at which the natural oil or fatty acid source is a liquid or molten, optionally adding a catalyst, then heating the reaction mixture until the desired esteramine, verified by acid value and alkalinity value, is reached. As used herein, “esteramine” is intended to encompass un-neutralized esteramine and esteramine in its neutralized (protonated), cationic salt form, unless the context clearly indicates otherwise.

The fatty acid source for preparing the esteramines can be a variety of starting materials, such as free fatty acids, fatty acid esters, or acid chlorides corresponding to fatty acids. The free fatty acids can be separate, such as a single purified fatty acid, or in combinations, such as fatty acid mixtures characteristic of the fatty acid constituents of glyceride esters in natural oils. Fatty acid esters can be glycerides, such as mono-, di- and/or triglycerides, or alkyl esters of fatty acids, such as methyl esters or ethyl esters of fatty acids. The fatty acid esters can be derived from a single fatty acid, or mixtures of fatty acids, such as those derived from natural fatty acid feedstocks or from natural oils.

In some embodiments, fatty acids, or alkyl ester derivatives thereof, are preferred over natural oils as the fatty acid source. Regardless of the fatty acid source, the resulting esteramine should have at least one alkyl chain having 10 carbon atoms or greater.

The esteramines may be prepared from C8-32 fatty acids, or alkyl ester derivatives thereof, that are saturated, unsaturated or a mixture of saturated and unsaturated fatty acids. In some embodiments, preferred fatty acids are those having carbon chain lengths of 16 to 20 carbon atoms. The fatty acids may be derived from various sources such as, for example, sunflower, canola, coconut, corn, cottonseed, flaxseed, peanut, meadowfoam, soybean, walnut, jojoba, palm, borage, safflower, rapeseed, tall oil, or mixtures thereof. In some embodiments, the fatty acids are derived from sunflower oil, canola oil or low erucic acid rapeseed oil (LEAR). In some embodiments, the fatty acids comprise at least 50% by weight, alternatively at least 60% by weight unsaturated fatty acid groups having at least one carbon-carbon double bond, and have an Iodine Value in the range of 40 to 130, preferably 50 to 130, more preferably 60 to 130.

The iodine value represents the mean iodine value of the parent fatty acyl compounds or fatty acids of all of the esterquat materials present. In the context of the present technology, the iodine value is defined as the number of grams of iodine which react with 100 grams of the parent compound. The method for calculating the iodine value of a parent fatty acyl compound/acid is known in the art and comprises dissolving a prescribed amount (from 0.1-3 g) into about 15 ml chloroform. The dissolved parent fatty acyl compound/fatty acid is then reacted with 25 ml of iodine monochloride in acetic acid solution (0.1M). To this, 20 ml of 10% potassium iodide solution and about 150 ml deionized water are added. After addition of the halogen has taken place, the excess of iodine monochloride is determined by titration with sodium thiosulfate solution (0.1M) in the presence of a blue starch indicator powder. At the same time a blank is determined with the same quantity of reagents and under the same conditions. The difference between the volume of sodium thiosulfate used in the blank and that used in the reaction with the parent fatty acyl compound or fatty acid enables the iodine value to be calculated.

The alkanolamines useful for preparing the esteramines correspond to the following general formula:

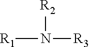

where R₁, R₂, and R₃ are independently selected from C₁₋₆ alkyl or hydroxy alkyl groups. Examples of alkanolamines include triethanol amine (TEA), methyl diethanolamine (MDEA), ethyl diethanolamine, dimethyl amino-N-(2,3-propanediol), diethylamino-N-(2,3-propanediol), methylamino-N,-N,-bis(2,3-propanediol), ethylamino-N,N-bis(2,3-propanediol), or mixtures thereof. In some embodiments, the alkanloamine comprises MDEA. In other embodiments, the alkanolamine comprises TEA. The molar ratio of fatty acid groups to alkanolamine is about 1.0:1 to about 2.2:1. In some embodiments, the alkanolamine is triethanolamine (TEA), and the molar ratio of fatty acid groups to TEA is about 1.3:1 to about 2.2:1, alternatively about 1.3:1 to 1.8:1. In other embodiments, the alkanolamine is MDEA, and the molar ratio of fatty acid groups to MDEA is about 1.0:1 to 2.0:1.

In some embodiments, it may be desirable to protonate the esteramine with an acid, thereby neutralizing the esteramine and forming an esteramine salt prior to absorbing it into the polysaccharide. The esteramine salt can be generated in-situ by reacting the corresponding esteramine with a sufficient amount of an acid to neutralize the esteramine to form the salt. The esteramine salt can have a pH in the range of about 2 to about 9, alternatively about 3 to about 7, alternatively about 3 to less than 7, alternatively about 3 to about 6, alternatively about 4 to about 6. In some embodiments, a stoichiometric amount of acid can be used for the neutralization. Alternatively, either an excess of acid or less than a stoichiometric amount of acid could be used, and less or more acid could then be added in the product formulation to adjust the pH of the final product to a desired level. Both organic and inorganic acids are suitable for in-situ reaction with an esteramine to generate the corresponding salts. Examples of acids include, but are not limited to, lactic acid, citric acid, maleic acid, adipic acid, boric acid, glutamic acid, glycolic acid, acetic acid, ascorbic acid, uric acid, oxalic acid, aspartic acid, butyric acid, lauric acid, glycine, formic acid, ethane sulfonic acid, hydrochloric acid, sulfuric acid, phosphoric acid, or combinations thereof.

### Amidoamines

Amidoamines used in the present technology can be prepared by reacting an amine with a fatty acid source. One preferred amine for reacting with the fatty acid source is diethylene triamine. Any of the fatty acid sources described above for preparing esteramines can be used to prepare the amidoamines. Regardless of the fatty acid source, the resulting amidoamine should have at least one alkyl chain having at least 10 carbon atoms or greater.

In some embodiments, the fatty acids have carbon chain lengths of 16 to 20 carbon atoms and comprise at least 50% by weight, alternatively at least 60% by weight unsaturated fatty acid groups having at least one carbon-carbon double bond, with an Iodine Value in the range of 40 to 130, preferably 50 to 130, more preferably 60 to 130. In some embodiments, the fatty acids are derived from sunflower oil, canola oil or low erucic acid rapeseed oil (LEAR).

Amidoamines can include, but are not limited to, alkylamidopropyl amines, alkylamidoethyl amines, or combinations thereof. Examples of amido amines that can be used are amidopropyl dimethyl amines, amidoethyl dimethyl amines, amidopropyl diethyl amines, diamidopropyl methylamines, diamidopropyl ethylamines, and diamidoethyl methylamines. Preferred amidoamines are those that are liquid at room temperature, preferably without a solvent, and particularly preferably without a VOC solvent.

In some embodiments, it may be desirable to protonate or neutralize the amine portion of the amidoamine with an acid, forming an amidoamine salt. Acids useful for neutralizing the amidoamine to form the salt include any of the acids described above for neutralizing an esteramine, such as lactic acid, citric acid, maleic acid, adipic acid, boric acid, glutamic acid, glycolic acid, acetic acid, ascorbic acid, uric acid, oxalic acid, aspartic acid, butyric acid, lauric acid, glycine, formic acid, ethane sulfonic acid, hydrochloric acid, sulfuric acid, phosphoric acid, or combinations thereof. A sufficient amount of acid is used to protonate the amidoamine, typically a stoichiometric or excess amount of acid. The amidoamine salt can have a pH in the range of about 2 to about 9, alternatively about 3 to about 7, alternatively about 3 to less than 7, alternatively about 3 to about 6, alternatively about 4 to about 6.

### Quaternary Ammonium Compounds

The quaternary ammonium compounds that can be used in the present technology include esterquats that are made by quaternizing any of the esteramines described above, quaternized amidoamines made by quaternizing any of the amidoamines described above, and quaternary ammonium compounds having alkyl, alkenyl, or aryl substituent groups, wherein the alkyl and alkenyl groups may be linear, branched or a combination thereof, bonded to the nitrogen atom. The alkyl, alkenyl, or aryl groups may be further derivatized with alcohol groups and alkoxylation, such as with ethylene oxide, propylene oxide, butylene oxide, or combinations of these.

### Esterquats

Methods of quaternizing tertiary esteramines to form esterquats are well known in the art. Quaternization of the esteramines is accomplished by reacting the esteramine with an alkylating agent, such as, for example, dimethyl sulfate, methyl chloride, diethyl sulfate, benzyl chloride, ethyl benzyl chloride, methyl bromide, or epichlorohydrin. The esterquats used in the present technology have at least one alkyl chain having 10 carbon atoms or greater. In one embodiment, the esterquat is a TEA-based esterquat having the following chemical structure:

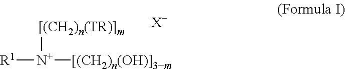

Each R is independently selected from a C5-31 alkyl or alkenyl group, alternatively a C7-21 alkyl or alkenyl group, alternatively a C19-21 alkyl or alkenyl group, alternatively an at least predominantly C13-17 alkyl or alkenyl group, and can be straight or branched. In some embodiments, the compounds of Formula I contain different R groups that are derived from a fatty acid material having an average Iodine Value of 60 to 130. R1 represents a C1-4 alkyl or hydroxyalkyl group or a C2-4 alkenyl group,

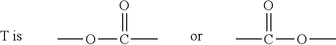

(i.e. a forward or reverse ester linkage); n is an integer selected from 0 to 4, alternatively from 2 to 4; m is 1 for a mono-esterquat, 2 for a di-esterquat, or 3 for a tri-esterquat, and denotes the number of moieties to which it refers that pend directly from the N atom, and X is an ionic group, such as a halide or alkyl sulfate, for example, a C1-4 alkyl or hydroxyalkyl sulfate or C2-4 alkenyl sulfate. Specifically contemplated anionic groups include chloride, methyl sulfate, or ethyl sulfate.

### Quaternized Amidoamines

Quaternized amidoamines used in the present technology are made by quaternizing any of the amidoamines described above with a suitable alkylating agent. Methods of quaternizing tertiary amidoamines are known in the art. Alkylating agents for the quaternization are also known, and can be any of the alkylating agents described above for quaternizing esteramines. The quaternized amidoamines used in the present technology have at least one alkyl chain having 10 carbon atoms or greater. Quaternized amidoamines are also commercially available from several sources. One particular example of a suitable quaternized amidoamine is ACCOSOFT® 780 PG, a methyl bis(canola amidoethyl)-2-hydroxyethyl ammonium methyl sulfate available from Stepan Company, Northfield, IL.

In some embodiments, the amount of unsaturated fatty acid groups in the esteramines, amidoamines, esterquats, or quaternized amidoamines may have an influence on the ability of the solid end product to disperse in water. Esteramines, amidoamines, esterquats, and quaternized amidoamines made from fatty acid feedstocks having an average Iodine Value of less than about 40 can result in solid compositions that are not easily dispersed in water.

### Other Quaternary Ammonium Compounds

Other quaternary ammonium compounds that may be used as the quaternary ammonium compound in the solid compositions of the present technology have the general formula:

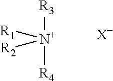

where R₁ is a straight or branched, saturated or unsaturated, alkyl or alkene chain having from 6 to 22, preferably from 8 to 18 carbon atoms;
R₂ is a straight or branched, saturated or unsaturated, alkyl or alkene chain having from 1 to 16 carbon atoms, preferably from 1 to 10 carbon atoms, with the proviso that at least one of R₁ or R₂ has an alkyl chain length of 10 carbon atoms or greater;
R₃ is methyl, ethyl, benzyl or ethylbenzyl;
R₄ is methyl or ethyl; and
X⁻ is: Cl⁻, Br⁻, F⁻, I⁻, (SO₄²⁻)_{1/2}, CH₃OSO₃⁻, HCO₃⁻, (CO₃²⁻)_{1/2}, CH₃COO⁻.

These quaternary ammonium compounds are useful, for example, as antimicrobial agents or fabric care agents. Exemplary quaternary ammonium compounds within the general formula include alkyl trimethyl ammonium halide, dialkyl dimethyl ammonium halide, alkyl dimethyl benzyl ammonium halide, dialkyl methyl benzyl ammonium halide, alkyl dimethyl ethylbenzyl ammonium halide, and dialkyl methyl ethylbenzyl ammonium halide. Specific quaternary ammonium salts include dialkyl dimethyl ammonium chloride (DDAC), such as didecyl dimethyl ammonium chloride, dioctyl dimethyl ammonium chloride, and octyl decyl dimethyl ammonium chloride, (C₁₂-C₁₈)-alkyl dimethyl benzyl ammonium chloride (ADBAC), (C₁₂-C₁₈)-alkyl dimethyl ethylbenzyl ammonium chloride, benzyltrimethyl ammonium chloride. The quaternary ammonium compound need not be a single entity, but may be a blend of two or more quaternary ammonium compounds.

The solid compositions of the present technology comprise from about 5% to about 70% by weight, alternatively about 5% to about 60% by weight, alternatively about 5% to about 55% by weight, alternatively about 10% to about 70% by weight, alternatively about 10% to about 60% by weight, alternatively about 10% to about 55% by weight, alternatively about 15% to about 70% by weight, alternatively about 15% to about 60% by weight, alternatively about 15% to about 55% by weight of the amine, protonated amine or quaternary ammonium compound active, based on the total weight of the composition.

### Polysaccharides

The solid compositions of the present technology also comprise from about 30% to about 95% by weight, alternatively from about 30% to about 80% by weight of a water-soluble polysaccharide. Suitable polysaccharides should be in a solid state at typical storage and use temperatures, and should be essentially non-chemically reactive with the other components in the composition. The polysaccharides should also be capable of absorbing the liquid or molten protonated amine or quaternary ammonium compound in amounts sufficient to obtain a high concentration of actives, such as 15% by weight or more, or 30% by weight or more, and desirably as high as 70% by weight actives. The polysaccharides should also be able to release the active when the solid composition is dispersed in water. Examples of suitable polysaccharides include maltodextrin, which can be derived from corn, rice, potato starch, oats, barley, rye, buckwheat, legumes or wheat, and agglomerated corn syrup solids.

In some embodiments, the maltodextrin is agglomerated maltodextrin. Ideally, agglomerated maltodextrin used in the present technology should have a particle size wherein 60% minimum passes through a 20 mesh screen and 15% maximum passes through a 200 mesh screen, preferably 70% minimum passes through a 20 mesh screen and 5% maximum passes through a 200 mesh screen. Other desirable properties of the agglomerated maltodextrin include a Dextrose Equivalent between 3 and 20, alternatively between 6 and 15, alternatively between 8 and 12; a moisture content below 10%, alternatively below 7.5%, alternatively below 5%, alternatively below 4%, and an aerated bulk density of less than 600 g/L, alternatively less than 350 g/L, alternatively less than 250 g/L, alternatively less than 200 g/L, and greater than about 100 g/L. In some embodiments, the agglomerated maltodextrin may have an aerated bulk density in the range of 100 g/L to less than 250 g/L. In other embodiments, the agglomerated maltodextrin may have an aerated bulk density of 250 g/L to about 600 g/L. Bulk densities of less than 250 g/L for the agglomerated maltodextrin tend to allow greater loadings of amine, protonated amine or quaternary ammonium compound active. Agglomerated maltodextrin and agglomerated corn syrup solids are commercially available from various sources, such as Grain Processing Corporation, Cargill, and Tereos. Surprisingly, in some embodiments, agglomerated maltodextrin has been found to enhance the water-dispersibility of the solid concentrated compositions.

### Solubilizers

Although not necessarily required, in some embodiments, it may be desirable to include a solubilizer in the solid composition formulation. The solubilizer acts to aid in the dissolution or flow of the liquid/flowable amine, protonated amine, or quat active so that the active can be better absorbed by the polysaccharide. Examples of solubilizers that could be used in the present technology include citric acid, sodium citrate, potassium carbonate, urea, sodium acetate and magnesium sulfate, or combinations thereof. A good solubilizer is one which has a high solubility in water such as above about 50 g per 100 g of deionized water, preferably above 70 g per 100 g of deionized water. WO 03/060053, incorporated herein by reference, describes desirable solubilizers (also called disintegrants) in detail. According to the Noyes-Whitney equation, the rate of dissolution is directly proportional to the saturation concentration of a given solute (solubilizer). Therefore, the higher the equilibrium, saturation concentration of a given solute is, the faster it will dissolve. When used, the amount of the solubilizer in the solid composition can range from about 0.5% to about 30%, alternatively about 1% to about 25%, alternatively about 2% to about 20%, alternatively about 3% to about 15% by weight of the composition.

### Optional Additional Ingredients

It is contemplated that the water-dispersible solid compositions can optionally comprise additional ingredients as desired or needed. Additional ingredients include, but are not limited to, nonionic surfactants, cationic surfactants, amphoteric surfactants, imidazolines, mercaptans, glycerides, glycerin, silicones, such as polydimethyl siloxane, amino silicones, or ethoxylated silicones, cationic polymers, or any combination thereof. Such additional ingredients can range from 0 to about 30% by weight, based on the total weight of the solid composition.

### Adjunct Ingredients

Adjunct ingredients may be added to the solid compositions of the present technology. The term “adjunct ingredient” includes: dispersing agents, stabilizers, pH control agents, antifoaming agents, metal ion control agents, colorants, brighteners, dyes, odor control agent, pro-perfumes, cyclodextrin, perfume, solvents, soil release agents, preservatives, antimicrobial agents, chlorine scavengers, anti-shrinkage agents, fabric crisping agents, spotting agents, anti-oxidants, anti-corrosion agents, bodying agents, drape and form control agents, smoothness agents, static control agents, wrinkle control agents, sanitization agents, disinfecting agents, germ control agents, mold control agents, mildew control agents, antiviral agents, drying agents, stain resistance agents, malodor control agents, fabric refreshing agents, chlorine bleach odor control agents, dye fixatives, dye transfer inhibitors, color maintenance agents, color restoration and rejuvenation agents, anti-fading agents, whiteness enhancers, anti-abrasion agents, wear resistance agents, fabric integrity agents, anti-wear agents, rinse aids, UV protection agents, sun fade inhibitors, insect repellents, anti-allergenic agents, enzymes, flame retardants, water proofing agents, fabric comfort agents, water conditioning agents, stretch resistance agents, hydrate inhibitors, scale inhibitors, demulsifiers, oxygen scavengers, and combinations thereof. The adjunct ingredients may be added to the solid compositions in an amount of 0 to about 3% by weight of the composition.

### Composition Properties

The solid compositions of the present technology are dispersible in water, and can be in the form of, for example, a powder, a tablet, pellets, a pouch, a pod, a packet, or a capsule. Powdered forms of the solid composition have a density of between 100 to 1050 g/liter, preferably, between 150 and 600 g/L, and are flowable. One method for measuring and calculating flowability is taught in the following reference: Peschl, I. & Colijn, H. 1976 “New Rotational Shear Testing Technique” in Bulk Solids Handling and Processing. Preferably, the compositions have a VOC content of less than 2%, and an aggregate BCI of at least 50.

### Methods of Making Solid Compositions

The solid compositions of the present technology can be prepared by adding the desired amount of liquid or molten amine, protonated amine, or quaternary ammonium compound to an appropriate amount of polysaccharide, and mixing until the amine, protonated amine, or quaternary ammonium compound is absorbed by the polysaccharide. If a solubilizer is used, it can be added before, with, or after the amine, protonated amine, or quaternary ammonium compound is mixed with the polysaccharide. Optional ingredients and adjunct ingredients may be added at any time.

### Product Use

The solid compositions of the present technology have a variety of uses. For example, in some embodiments, the solid composition can be a solid fabric softener composition that can be used, for example, in the rinse cycle of a home washing machine. The solid composition can be added directly, without dilution, for example through a dispenser drawer or, for a top-loading washing machine, directly into the drum. The compositions can also be used, for example, for hair conditioning or hair repair applications, disinfectant or sanitizer applications, or in oilfield applications, including oil and gas transport, production, stimulation, and reservoir conformance. In some embodiments, the solid compositions are in a powder form that can be dispensed by scooping or shaking the powder from a product container. Alternatively, the solid compositions can be encapsulated within a water-soluble or water-rupturable coating or film to form, for example, a pod, a packet, a pouch, or a capsule. In other embodiments, the solid composition can be pressed into tablets or other forms, such as pellets, pastilles, sticks or pucks. The pressed forms or encapsulates can contain a unit dose of the solid composition. The term “unit dose” as used herein refers to a pre-metered amount of the solid composition that should be delivered to provide a particular result upon dispersion or dilution in a liquid. Water-soluble or water-rupturable coatings or films are known in the art. Suitable materials for the coating or film include, but are not limited to, polyvinyl alcohol, polyvinyl pyrrolidone, methyl cellulose, hydroxymethyl cellulose, partially hydrolyzed vinyl acetate, gelatins, and combinations thereof.

Alternatively, the solid compositions can be diluted prior to use, preferably with water, to an appropriate concentration of the amine, protonated amine or quaternary ammonium active to achieve a desired results. For example, when the solid composition is formulated as a fabric softener, the solid composition can be diluted in water to a concentration of about 2% to about 22% by active weight, preferably about 3% to about 8% by active weight, based on the total weight of the diluted composition. Since embodiments of the solid composition are easily dispersed in water, it is contemplated that the dilution could be done by a consumer. Such use provides several advantages, such as reduced packaging needs (due to the concentrated product) and reduced energy needs for transportation, as well as reduced transportation costs, due to less water needing to be shipped.

The compositions of the present technology have a variety of end uses, and can be formulated into a variety of end use products. Examples of specific end use products in which the solid compositions can advantageously be used include, but are not limited to, hair conditioners, hair repair compositions, fabric softeners, fabric conditioners, pool sanitizers, hard surface disinfectants, and corrosion inhibitors.

End use product formulations comprising the solid composition may contain other optional ingredients suitable for use, such as surfactants or other additives, and a diluent, such as water. Examples of surfactants include nonionic, cationic, and amphoteric surfactants, or combinations thereof. Examples of nonionic surfactants include, but are not limited to, fatty alcohol alkoxylates, polyalkylene glycols, mono- and/or dialkyl sulfosuccinates, fatty acid isethionates, fatty acid sarcosinates, fatty acid glutamates, ether carboxylic acids, alkyl oligoglucosides, and combinations thereof. Examples of cationics include, but are not limited to, behentrimonium chloride (BTAC), cetrimonium chloride (CETAC), and polyquaterniums, or combinations thereof. Examples of amphoteric surfactants include, but are not limited to, betaines, amidopropylbetaines, or combinations thereof. Surfactant amounts in the product formulation can range from about 0.01% to about 10% by weight of the end product formulation.

Examples of additives include rheological modifiers, emollients, skin conditioning agents, emulsifier/suspending agents, fragrances, colors, herbal extracts, vitamins, builders, enzymes, preservatives, antibacterial agents, or combinations thereof. For some product formulations, pH adjusters can be added to adjust the pH of the formulation to a pH in the range of about 1.5 to about 8.0, alternatively about 2.0 to about 6.5. Examples of pH adjusters that can be used include any of the acids mentioned above for protonating the esteramines or amidoamines. Total additives in the product formulation can range from about 0.01% to about 10% by weight of the end product formulation.

The solid compositions of the present technology provide several benefits. Since the compositions have no or minimal water, preservatives may not be necessary to include in the composition, or can be used in lower amounts. The low or minimal amount of water also contributes to increased product stability, particularly when the amine or quaternary ammonium compound has ester linkages, since no hydrolysis occurs in the absence of water. Having low or minimal water can also reduce the packaging requirements, as well as reducing shipping costs, due to less water weight. Packaging for the solid compositions could be cardboard, which is recyclable and biodegradable, lighter in weight than plastics, and potentially reduces the amount of microplastics introduced into the environment, making the solid products more environmentally friendly. The solid compositions are also non-flammable, and afford the ability to incorporated high levels of perfume, such as above 2 percent by weight.

### EXAMPLES

The presently described technology and its advantages will be better understood by reference to the following examples. These examples are provided to describe specific embodiments of the present technology. By providing these examples, the inventors do not limit the scope and spirit of the present technology.

### Example 1

An esterquat was made as follows-canola fatty acid (283 g/mol, 2876.0 g, 10.2 mol) and Antioxidant 1010 (1178 g/mol, 3.7 g, 0.003 mol) were added to a 5 L reactor equipped with mechanical stirring, nitrogen sparge and distillation capabilities. Stirring was initiated, the contents were heated to 35° C. and triethanolamine (149 g/mol, 977.03 g, 6.5 mol) was added. The reaction temperature was increased to 190° C. and held for 3.5 hr. After 3.5 hr, the reactor was cooled and the esteramine intermediate was transferred for quaternization and tested (Free Amine=1.77 meq/g, Total Acidity=0.06 meq/g).

The esteramine intermediate (564 g/mol, 3650.3 g, 6.5 mol) was added to a 5 L reactor equipped with mechanical stirring, nitrogen headspace sweep and reflux capabilities. Stirring and nitrogen sweep were initiated. The reaction temperature was adjusted to 50° C. and dimethyl sulfate (126 g/mol, 774.8 g, 6.1 mol) was added drop wise over one hour. Temperature was controlled to 85° C. max during the addition. Reaction was mixed for 1 hr at 85° C. Sodium chlorite, 25% (wt) (90.4 g/mol, 9.8 g, 0.03 mol) was added and mixed for 30 min. Product was collected and tested (Free Amine=0.08 meq/g, Cationic Actives=1.17 meq/g, Total Acidity=0.10 meq/g, Gardner Color=4.6). A slightly yellow paste was obtained. This esterquat is designated EQ1.

### Example 2

Canola fatty acid (283 g/mol, 647.8 g, 2.3 mol), triethanolamine (149 g/mol, 171.0 g, 1.1 mol) and Antioxidant 1010 (1178 g/mol, 0.82 g, 0.001 mol) were added to a 2 L reactor equipped with mechanical stirring, nitrogen sub-surface sparge and distillation capabilities. Stirring was initiated and the contents were heated to 75° C. Nitrogen sparge was started. The reaction temperature was then increased to 190° C. and held for 4.5 hr. After 4.5 hr, the reactor was cooled and the esteramine intermediate was transferred for quaternization and tested (Free Amine=1.48 meq/g, Total Acidity=0.05 meq/g).

The esteramine intermediate (675 g/mol, 753.7 g, 1.1 mol) was added to a 2 L reactor equipped with mechanical stirring, nitrogen headspace sweep and reflux capabilities. Stirring and nitrogen sweep were initiated. The reaction temperature was adjusted to 45° C. Dimethyl sulfate (126 g/mol, 130.5 g, 1.0 mol) was added drop wise over one hour. Temperature was controlled to 85° C. max during the addition. Reaction was mixed for 1 hr at 85° C. Product was collected and tested (Free Amine=0.09 meq/g, Cationic Actives=1.16 meq/g, Total Acidity=0.01 meq/g). A slightly yellow paste was obtained. This esterquat is designated EQ2.

### Example 3

Distilled tallow fatty acid (272 g/mol, 1067.05 g, 3.9 mol) and hydrogenated tallow fatty acid (272 g/mol, 409.89 g, 1.5 mol) were added to a 3 L reactor equipped with mechanical stirring, nitrogen sub-surface sparge and distillation capabilities. The iodine value of this fatty acid mixture is about 34. Stirring was initiated and the contents were heated to 75° C. Triethanolamine (149 g/mol, 521.3 g, 3.5 mol), Antioxidant 1010 (1178 g/mol, 2.0 g, 0.002 mol) and phosphorous acid (82 g/mol, 1.0 g, 0.01 mol) were added. Nitrogen sparge was started. The reaction temperature was then increased to 190° C. and held for 4 hr. After 4 hr, the reactor was cooled and the esteramine intermediate was transferred for quaternization and tested (Free Amine=1.81 meq/g, Total Acidity=0.06 meq/g).

The esteramine intermediate (552 g/mol, 1836.0 g, 3.3 mol) was added to a 3 L reactor equipped with mechanical stirring, nitrogen headspace sweep and reflux capabilities. Stirring and nitrogen sweep were initiated. The reaction temperature was adjusted to 45° C. Dimethyl sulfate (126 g/mol, 381.8 g, 3.0 mol) was added drop wise over 30 minutes. Temperature was controlled to 85° C. max during the addition. Reaction was mixed for 1 hr at 85° C. Dimethyl sulfate (126 g/mol, 20.0 g, 0.2 mol) was added drop wise. Temperature was controlled to 85° C. max during the addition. Reaction was mixed for 1 hr at 85° C. Product was collected and tested (Free Amine=0.08 meq/g, Cationic Actives=1.16 meq/g, Total Acidity=0.17 meq/g). A waxy solid was obtained. This esterquat is designated EQ3.

### Formulation General Procedure

The example formulations made with agglomerated maltodextrin were processed in the following general manner: the desired amount of agglomerated maltodextrin is added to a mixing vessel, liquid quaternary ammonium compound or protonated amine is added to the vessel with gentle mixing until all the liquid has been added, optionally, solubilizer is then added. Mixing is stopped when the liquid is fully absorbed and the product is homogeneous.

### Fabric Softener Examples

### Example 4

A solid concentrated composition was made according to the general procedure and comprised 47.5% by weight EQ1, 47.5% by weight agglomerated maltodextrin (AMD), (Maltrin M700 from Grain Processing Corporation), and 5% by weight anhydrous citric acid. The density of this composition was measured to be 160 g/L.

### Example 5

A solid concentrated composition was made according to the general procedure and comprised 55% by weight EQ1, 35% by weight AMD, and 10% by weight anhydrous citric acid.

### Example 6

A solid concentrated composition was made according to the general procedure and comprised 47.5% by weight EQ2, 47.5% by weight AMD, and 5% by weight anhydrous citric acid. EQ2 differs from EQ1, used in Examples 4 and 5, in that EQ2 has a fatty acid to TEA ratio of 2.00:1, whereas EQ1 has a fatty acid to TEA ratio of 1.55:1.

### Example 7

A solid concentrated composition was made according to the general procedure and comprised 47.5% by weight EQ3, 47.5% by weight AMD and 5% by weight anhydrous citric acid. EQ3 differs from EQ1, used in Examples 4 and 5, in that EQ3 is made from a tallow fatty acid feedstock having an iodine value of 34, rather than the canola fatty acid feedstock used to make EQ1.

### Example 8

In this example, the solid concentrated compositions of Examples 4-7 were assessed for dispersibility of the solid composition in water using the following test: about 0.5 grams of the formula was added to an 8 ounce jar containing 120 ml of water, the solution was then mixed with a tongue depressor by hand for 10 seconds at ambient temperature. If there were no visibly discreet particles after mixing, the formula was deemed to be dispersible. The results are shown in Table 1.

TABLE 1

| Example | Physical Condition |
|---|---|
| Example 4: 47.5/47.5/5 EQ1/AMD/citric | Free flowing and dispersible |
| Example 5: 55/35/10 EQ1/AMD/citric | Free flowing and dispersible |
| Example 6: 47.5/47.5/5 EQ2/AMD/citric | Not dispersible |
| Example 7: 47.5/47.5/5 EQ3/AMD/citric | Not dispersible |

The results in Table 1 show that the Example 6 composition, made with EQ2, was not readily dispersible in water, whereas the compositions of Examples 4 and 5, made with EQ1, were dispersible. The fatty acid to TEA ratio for EQ1 is 1.55:1 and for EQ2, the ratio is 2.00:1. These results indicate that the dispersibility of the solid composition in water may be influenced by the fatty acid to TEA ratio used in making the esterquat. The results show that, when using a canola fatty acid-based esterquat (TEA/DMS) in the solid concentrated composition, the ratio of the fatty acid groups to TEA should be below 2.0:1 to obtain a dispersible composition. The results in Table 1 also show that the Example 7 composition, made with EQ3, was not readily dispersible. The results indicate that the dispersibility of the solid composition in water may also be influenced by the iodine value of the fatty acid feedstock used in making the esterquat. These results show that the iodine value of the fatty acid feedstock used to make esterquat should be above 34 to obtain a dispersible solid composition.

### Example 9

This example evaluates the softening ability of solid compositions according to the present technology. Softening tests were run using the following methodology based on ASTM D-5237: white hand towels made from an 86/14 cotton/polyester blend were first subjected to a prewash process to remove any factory finish. For each test, 160 towels were washed in conventional household washing machines. Experimental fabric softener samples were dosed into the machines during the rinse cycle. Towels were then tumble dried and allowed to equilibrate to room temperature overnight. Panelists then blindly evaluated pairs of towels via a Paired Comparison panel test. The number of votes were tallied for each sample. Using the One-Sided Directional Difference Test (Meilgaard, M. C., Civille, G. V., Carr, B. T., Sensory Evaluation Techniques, 3rd Ed., CRC Press, 1999, pp. 277-278, 355, 371), in a 160-vote observation test one product would need to be chosen a minimum of 91 times to be deemed statistically superior to the other at the 95% confidence level.

Using this test method, the composition of Example 4 had softening equivalent to a 5% traditional liquid fabric softener made from the same esterquat and dosed at the same amount of active into the rinse cycle of a washing machine. The composition of Example 5 afforded superior softening compared to that of a 5% traditional liquid fabric softener made from the same esterquat and dosed at the same amount of active into the rinse cycle of a washing machine.

### Example 10

In order to determine if the maltodextrin needs to be pre-agglomerated, the following experiment was run: 100 g of maltodextrin (not pre-agglomerated) and 10.5 g of anhydrous citric acid were added to the bowl of an eight cup consumer grade food processor. The food processor was turned on low and, while mixing, EQ1 was added. The speed was intermittently set to high for 10 seconds at a time in an attempt to agglomerate the particles. After about 45 g of EQ1 was added, the batch became too sticky and clumpy and the experiment was stopped. This example demonstrates that the maltodextrin needs to be pre-agglomerated when the concentration of the active cationic material is greater than about 25% in the composition. By repeating the experiment, but adding lesser amounts of EQ1, it was determined that 15% by weight EQ1 on non-agglomerated maltodextrin results in a free-flowing composition that disperses in water. Using 20% by weight EQ1 on non-agglomerated maltodextrin results in a composition that flows but does not disperse in water. A composition is considered to be flowable if, when a container such as a cardboard box it is tipped over, all the small particles readily fall out of the container, remain discrete small particles which do not stick together or form clumps, and no residue is left in the bottom of the container. Any type container can be used to run this flowability test.

### Example 11

Using a series of different solvents per the method described in the book Solubility Science, Principles and Practice, Steven Abbott, 2017, Creative Common NY-BD, hereby incorporated by reference, the Hansen polarity parameter for EQ1 was measured to be 10.9 while the Hansen polarity parameter of EQ3 was measured to be 4.4. This shows that in order for the composition to disperse in water, the Hansen polarity parameter of the EQ should be above about 5.

### Example 12

A solid composition was made according to the general procedure and comprised 60% by weight AMD and 40% by weight EQ1. The composition is flowable, and dispersibility testing showed that the composition disperses in water.

### Example 13

A solid composition was made according to the general procedure, and comprised 55% by weight of methyl bis(canola amidoethyl)-2-hydroxyethyl ammonium methyl sulfate, an amidoamine-based softening quat made using canola oil and diethylenetriamine (ACCOSOFT® 780 PG, available from Stepan Company), 35% by weight AMD, and 10% anhydrous citric acid. The composition is flowable, and dispersibility testing showed that the composition disperses in water. The composition was also evaluated for softening ability using the test procedure described above. Testing showed that the composition provided equivalent softening to that of a 5% traditional liquid fabric softener made from the same amidoamine-based quat and dosed at the same amount of active into the rinse cycle of a washing machine.

### Example 14

The composition of Example 4 was made into a tablet by adding approximately 0.5 g of the powdered formula in a tablet press, compressing the material for 10 seconds with the lever and then carefully removing the tablet. Many tablets can be made using this process.

### Example 15

A solid concentrated composition was made according to the general procedure and comprised 30% by weight EQ1, 65% by weight agglomerated maltodextrin 2 (AMD2; Maltrin M500 from Grain Processing Corporation), and 5% by weight anhydrous citric acid. The density of this composition was measured to be 490 g/L.

### Example 16

A solid concentrated composition was made according to the general procedure and comprised 40% by weight EQ1, 55% by weight agglomerated maltodextrin 2, and 5% by weight anhydrous citric acid. It was observed this formulation was too sticky/not flowable enough, indicating that this agglomerated maltodextrin was not able to absorb all of the EQ1. Agglomerated maltodextrin 2 has an aerated bulk density of about 500 g/L, whereas the agglomerated maltodextrin used in Examples 4-9 and 12-13 has an aerated bulk density of about 150 g/L. This example demonstrates that the density of the agglomerated polysaccharide may have an effect on the amount of quat or amine that can be absorbed by the polysaccharide. For higher loadings of quat or amine, i.e. about 35% by weight or more of the solid composition, the aerated bulk density of the agglomerated polysaccharide should be in the range of about 100 g/L to less than about 250 g/L. When the solid composition comprises an agglomerated polysaccharide having an aerated bulk density of 250 g/L or higher, the amount of quat or amine used in the composition should be less than about 35% by weight to insure adequate absorption by the polysaccharide.

The composition of this example and that of Example 15 were compared with a Freeman Technology FT4 Powder rheometer utilizing the shear cell setup. The methodology used in this evaluation is explained in detail on the Freeman Technology website: www.freemantech.co.uk. The evaluation subjected an 85 mL sample of each composition to pre-shearing at 9 kPa normal stress before subsequent shear tests were carried out at 7, 6, 5, 4, and 3 kPa normal stresses. The flowability, REL (p), and cohesion (in kPa) of each composition was measured; flowability was measured and calculated as taught in the following reference, which is hereby incorporated by reference: Peschl, I. & Colijn, H. 1976 “New Rotational Shear Testing Technique” in Bulk Solids Handling and Processing. Results are shown in Table 2.

TABLE 2

| Composition | REL(p) | Cohesion (kPa) |
|---|---|---|
| Example 15 | 2.30 | 1.55 |
| Example 16 | 1.77 | 2.23 |

Based on the results, it is desirable to have the flowability parameter above about 2.0 and the cohesion parameter below about 2.0 for the composition to flow properly and not stick together too much.

### Hair Conditioner Examples

### Example 17

A solid composition was made according to the general procedure, and comprised 30% by weight of an esterquat/glycerides mixture and 70% by weight AMD. The esterquat/glycerides mixture comprised about 70% by weight of an esterquat derived from sunflower oil reacted with TEA, and about 30% by weight of glycerides. The composition is flowable, and dispersibility testing showed that the composition disperses in water.

### Example 18

A solid composition was made by melting BTAC (Genamin BTLF from Clariant), and mixing the molten BTAC with AMD according to the general procedure. The composition comprised 30% by weight of BTAC and 70% by weight AMD and was flowable. Dispersibility testing showed that the composition disperses in water.

### Example 19

A solid composition was made by mixing 15% by weight of an esterquat/glycerides mixture (70% esterquat/30% glycerides) and 85% by weight of non-agglomerated maltodextrin until the esterquat/glycerides mixture was fully absorbed. The resulting solid composition is a free-flowing powder but does not easily disperse in water when wetted and rubbed between hands—it formed small, gel-like balls that took a while to dissolve. A comparison of the results of this example with the results of Example 17 shows that dispersibility of the solid composition can be improved by using agglomerated maltodextrin as the polysaccharide. Also, a comparison of the results of this example with the results of Example 10, which used 15% by weight EQ1 rather than an esterquat/glyceride mixture, shows that the presence of glycerides in the solid composition of this example may affect the dispersibility of the solid composition.

### Oilfield Compositions

### Example 20

A solid composition for use as a corrosion inhibitor was prepared by adding 5.05 g of agglomerated maltodextrin (Maltrin M700) to a 20 ml scintillation vial at ambient temperature, followed by the addition of 5.01 g of a general corrosion inhibitor composition. The general corrosion inhibitor comprised 80% by active weight of a combination of 47% by weight imidazoline (tall oil fatty acid/diethylenetriamine acetic acid salt), 20% by weight ADBAC, and 13% by weight mercaptoethanol, in 20% by weight solvent, which comprised equal parts of water and methanol. The vial was capped and the contents were vigorously shaken by hand for 20 seconds. This composition resulted in a flowing powder with 40% active corrosion inhibitor. This sample was then further diluted to a 20% active solution in deionized water for corrosion inhibition evaluation.

A Rotating Cylinder Electrode (RCE) test was utilized to evaluate the performance of the general corrosion inhibitor with the maltodextrin for corrosion inhibition application. In this test, the corrosion rate is measured by an electrochemical technique called Linear Polarization Resistance (LPR) with a working electrode, a platinum counter electrode, and a standard calomel reference electrode. The experiment is run at low shear rate, atmosphere pressure, temperature up to 80° C., and under sweet, carbon dioxide, conditions. The corrosion data is measured and monitored by Gamry potentiostat and Gamry Framework software is used for analysis.

For the test, a glass cell was loaded with 700 g of brine solution (3.5% NaCl, 0.11% CaCl₂)*2H₂O, 0.07% MgCl₂*6H₂O) and warmed to 80° C. under carbon dioxide sparging for two hours. The metal coupon (C1018) was rotated at 3000 rpm. After establishing a baseline corrosion rate for 4 hours, the 20% active corrosion inhibitor solution was injected at 25 ppm dosage (18 μL), and the corrosion rate profile was monitored for a total test time of 20 hours.

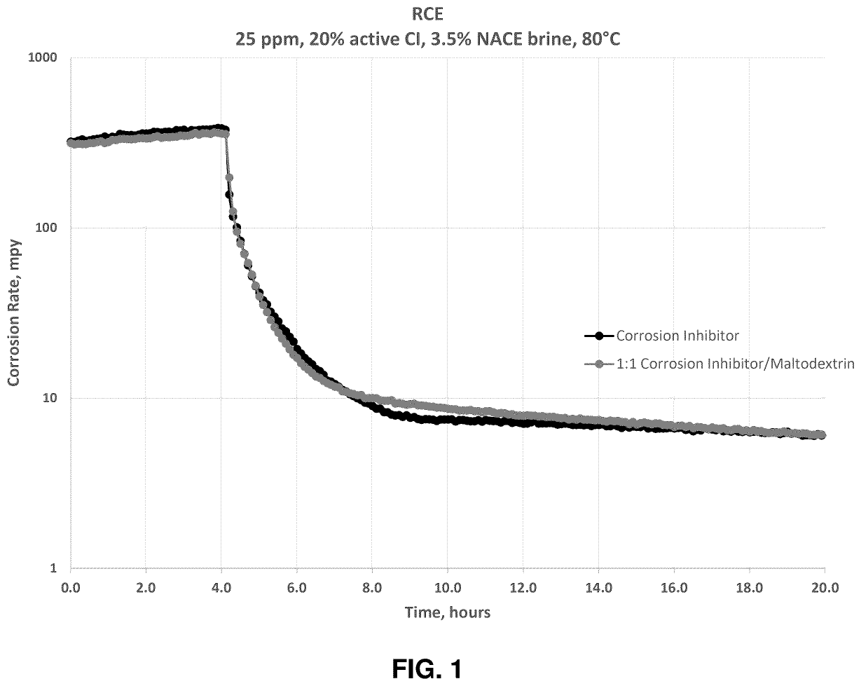

FIG. 1 shows the corrosion rate profile of the general corrosion inhibitor alone and combined with maltodextrin. It can be seen that the addition of maltodextrin does not affect the corrosion rate of the corrosion inhibitor.

### Example 21

A solid composition is prepared by adding a TEA-based coco dibutyl esterquat to agglomerated maltodextrin at ambient temperature, and mixing until the esterquat is absorbed. The solid composition is a free-flowing powder. The solid composition could be used as a hydrate inhibitor in oilfield applications.

### Antimicrobial Compositions

### Example 22

A solid composition for use as an antimicrobial composition was prepared by mixing equal parts by weight of agglomerated maltodextrin (MALTRIN M700 from Grain Processing Corporation) and n-alkyl dimethyl benzyl ammonium chloride (BTC® 8358 from Stepan Company, Northfield, Illinois) at ambient temperature until the quaternary ammonium compound was absorbed. The resulting composition is a free-flowing powder, even though the composition contains 10% water coming from the BTC® 8358 product. The solid composition contains 40% active weight of the biocidal quaternary ammonium compound.

### Example 23

Solid compositions for use as antimicrobial compositions were prepared by mixing different quaternary ammonium compounds with agglomerated maltodextrin at ambient temperature until the quaternary ammonium compound was absorbed. The composition prepared and the physical characteristics of each composition are shown in Table 3.

| | | | Wt % | | |
|---|---|---|---|---|---|
| | | Wt % | Quat | Wt % | Physical |
| Composition | Quat | Quat | Actives | Maltodextrin | Characteristics |
| 1 | BTC ® 2125 | 50% | 50% | 50% | Mixture clumps |
| | base (91-95% | | | | together |
| | active quat) | | | | |
| 2 | BTC ® 2125 | 40% | 40% | 60% | Powder |
| | base (91-95% | | | | |
| | active quat) | | | | |
| 3 | BTC ® 1010 | 62.5% | 50% | 37.5% | Slurry |
| | (80% active | | | | |
| | quat) | | | | |
| 4 | BTC ® 1010 | 40% | 32% | 60% | Powder |
| | (80% active | | | | |
| | quat) | | | | |

BTC ® 2125 Base: Mix of n-alkyl dimethyl ammonium chloride and n-alkyl dimethyl ethyl benzyl ammonium chloride without solvents or inerts (91-95% active quat, 4.28% water, 2.3% max combined free amine and amine HCl)
BTC ® 1010: Didecyl-dimethyl ammonium chloride (80% active quat)

The results in Table 3 show that Composition 1, comprising 50% by weight of active BTC® 2125 quat is a sticky mixture, whereas Composition 2, using the same quat, but at 40% active quat, is a powder. Similarly, Composition 3, comprising 50% by weight of active BTC® 1010 is a slurry, whereas Composition 4, at 32% by weight active, is a powder. These results show that solid powder compositions can be prepared which contain up to 50% by active weight of the biocidal quat. While a dry free-flowing powder is preferred, the higher active clumpy material is acceptable for compressing into a water dispersible tablet, pastille, stick or puck.

The compositions are assessed for antimicrobial efficacy, and it is determined that the addition of maltodextrin does not negatively impact the antimicrobial efficacy of the biocidal quaternary ammonium compound.

The present technology is now described in such full, clear and concise terms as to enable a person skilled in the art to which it pertains, to practice the same. It is to be understood that the foregoing describes preferred embodiments of the present technology and that modifications may be made therein without departing from the spirit or scope of the present technology as set forth in the appended claims. Further, the examples are provided to not be exhaustive but illustrative of several embodiments that fall within the scope of the claims.

1. A water-dispersible solid composition comprising:
(a) from about 5% to about 70% by weight of at least one quaternary ammonium compound, amine or protonated amine, or mixtures thereof; wherein the quaternary ammonium compound is selected from (i) an esterquat that is the quaternized reaction product of a fatty acid source having an Iodine Value of 40 to 130 reacted with triethanolamine (TEA) at a fatty acid to alkanolamine molar ratio of 1.3:1 to 1.8:1, or reacted with methyl diethanolamine (MDEA) at a fatty acid to alkanolamine molar ratio of 1.0:1 to 2.0:1, (ii) an amidoamine quat, or (iii) a quaternary ammonium compound having the following chemical formula: 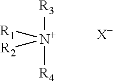
where R₁ is a straight or branched, saturated or unsaturated, alkyl or alkene chain having from 6 to 22, preferably from 8 to 18 carbon atoms;
R₂ is a straight or branched, saturated or unsaturated, alkyl or alkene chain having from 1 to 16 carbon atoms, preferably from 1 to 10 carbon atoms;
R₃ is methyl, ethyl, benzyl, or ethylbenzyl;
R₄ is methyl or ethyl; and
X⁻ is: Cl⁻, Br⁻, F⁻, I⁻, (SO₄²⁻)_{1/2}, CH₃OSO₃⁻, HCO₃⁻, (CO₃²⁻)_{1/2}, or CH₃COO⁻; and wherein the amine or protonated amine is selected from an esteramine, an amidoamine, or an amidoamine salt;
(b) from about 30% to about 95% by weight of water-soluble polysaccharide having an aerated bulk density in the range of 100 g/L to 600 g/L, wherein the water-soluble polysaccharide comprises maltodextrin or agglomerated maltodextrin; and
(c) optionally, up to 30% by weight of a solubilizer;
wherein,
the quaternary ammonium compound, amine or protonated amine has at least one alkyl chain of 10 carbons or greater, and is absorbed by the polysaccharide; with the proviso that, when the water-soluble polysaccharide is not agglomerated, the amount of the quaternary ammonium compound, amine, or protonated amine in the solid composition is not greater than 20% by weight, and when the water-soluble polysaccharide is agglomerated and has an aerated bulk density in the range of 250 g/L to 600 g/L, the amount of the quaternary ammonium compound, amine, or protonated amine in the solid composition is not greater than 35% by weight.

2. The composition of claim 1, wherein the at least one amine, protonated amine or quaternary ammonium compound is at least one esteramine or esterquat.

3. The composition of claim 1, wherein the fatty acid source is derived from sunflower oil, canola oil, low erucic acid rapeseed (LEAR) oil, or a combination thereof.

4. The composition of claim 1, wherein the quaternary ammonium compound is an esterquat having a Hansen polarity parameter of greater than about 5.

5. The composition of claim 1, wherein the at least one amine, protonated amine or quaternary ammonium compound is at least one amidoamine or amidoamine salt.

6. The composition of claim 1, wherein the solubilizer is selected from the group consisting of citric acid, sodium citrate, potassium carbonate, urea, sodium acetate, magnesium sulfate, and combinations thereof.

7. The composition of claim 1, wherein the solid composition is in the form of a powder.

8. The composition of claim 1, wherein the solid composition has a flowability parameter value above about 2.0 and a cohesion parameter value below about 2.0.

9. The composition of claim 1, wherein the solid composition is in the form of a pod, a packet, a pouch, or a capsule.

10. The composition of claim 1, wherein the solid composition is in the form of a tablet, a stick, a puck, pastilles, or pellets.

11. A method of making a water-dispersible solid composition comprising the steps of:
providing a water-soluble polysaccharide in an amount of about 30% to about 95% by weight, based on the total weight of the solid composition, wherein the water-soluble polysaccharide comprises maltodextrin or agglomerated maltodextrin;
adding from about 5% to about 70% by weight of at least one liquid or molten amine, protonated amine, or quaternary ammonium compound, or mixtures thereof, to the water-soluble polysaccharide, wherein the quaternary ammonium compound is selected from (i) an esterquat that is the quaternized reaction product of a fatty acid source having an Iodine Value of 40 to 130 reacted with triethanolamine (TEA) at a fatty acid to alkanolamine molar ratio of 1.3:1 to 1.8:1, or reacted with methyl diethanolamine (MDEA) at a fatty acid to alkanolamine molar ratio of 1.0:1 to 2.0:1, (ii) an amidoamine quat, or (iii) a quaternary ammonium compound having the following chemical formula: 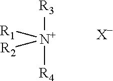
where R₁ is a straight or branched, saturated or unsaturated, alkyl or alkene chain having from 6 to 22, preferably from 8 to 18 carbon atoms;
R₂ is a straight or branched, saturated or unsaturated, alkyl or alkene chain having from 1 to 16 carbon atoms, preferably from 1 to 10 carbon atoms;
R₃ is methyl, ethyl, benzyl, or ethylbenzyl;
R₄ is methyl or ethyl; and
X⁻ is: Cl⁻, Br⁻, F⁻, I⁻, (SO₄²⁻)_{1/2}, CH₃OSO₃⁻, HCO₃⁻, (CO₃²⁻)_{1/2}, or CH₃COO⁻; and wherein the amine or protonated amine is selected from an esteramine, an amidoamine, or an amidoamine salt;
with the proviso that, when the water-soluble polysaccharide is not agglomerated, the amount of the amine, protonated amine, or quaternary ammonium compound added to the water-soluble polysaccharide is not greater than 20% by weight of the solid composition, and when the water-soluble polysaccharide is agglomerated, and has an aerated bulk density in the range of 250 g/L to 600 g/L, the amount of the amine, protonated amine, or quaternary ammonium compound added to the agglomerated polysaccharide is not greater than 35% by weight of the solid composition; and
mixing the water-soluble polysaccharide and the amine, protonated amine or quaternary ammonium compound until the amine, protonated amine or quaternary ammonium compound is absorbed by the water-soluble polysaccharide to form the water-dispersible solid composition.

12. The method of claim 11, further comprising the step of adding from about 0.5% to about 50% by weight of a solubilizer before, during, or after the addition of the protonated amine or quaternary ammonium compound.

13. The method of claim 11, wherein the at least one amine, protonated amine, or quaternary ammonium compound is at least one esteramine or esterquat.

14. The method of claim 11, wherein the fatty acid source is derived from sunflower oil, canola oil, LEAR oil, or a combination thereof.

15. The method of claim 11, wherein the quaternary ammonium compound is an esterquat having a Hansen polarity parameter of greater than 5.

16. The method of claim 11, wherein the at least one amine, protonated amine, or quaternary ammonium compounds is at least one amidoamine or amidoamine salt.

17. The method of claim 12, wherein the solubilizer is selected from the group consisting of citric acid, sodium citrate, potassium carbonate, urea, sodium acetate, magnesium sulfate, and combinations thereof.

18. The method of claim 11, wherein the solid composition is in the form of a powder.

19. The method of claim 11, wherein the powder is encapsulated within a water-soluble or water-rupturable film or coating.

20. The method of claim 11, wherein the solid composition is formed into a tablet, a stick, a puck, pastilles, or pellets.
